(19) United States
(12) Patent Application Publication
(10) Pub. No.: US 2016/0216508 A1
(43) Pub. Date: Jul. 28, 2016

(54) ACTUATOR AND VARIABLE SHAPE MIRROR USING ACTUATOR

(71) Applicant: CANON KABUSHIKI KAISHA, Tokyo (JP)

(72) Inventor: Kenji Tamamori, Ebina-shi (JP)

(21) Appl. No.: 14/980,883

(22) Filed: Dec. 28, 2015

(30) Foreign Application Priority Data
Jan. 27, 2015 (JP) 2015-013791

(51) Int. Cl. G02B 26/08; A61B 3/10; H02N 1/00; G02B 27/00

(52) U.S. Cl. CPC G02B 26/0841; G02B 27/0068; A61B 3/1015; H02N 1/008

(57) ABSTRACT

Provided is an actuator having a structure with which stress concentration on an elastic member of the actuator can be reduced in a manufacturing process thereof and breakage of the elastic member can be inhibited. The actuator includes a movable member, an elastic member configured to connect the movable member and a supporting member to each other, and an electrode pair having a comb electrode structure for displacing the movable member in a direction perpendicular to a reflective surface in which all movable comb electrodes extending from the movable member are substantially in parallel with one another, and a portion of the elastic member, which is located at a beginning of extension from the movable member, is substantially in parallel with the movable comb electrodes.

### BACKGROUND OF THE INVENTION

1. Field of the Invention

The present invention relates to an actuator, a variable shape mirror using the actuator, an apparatus using the variable shape mirror, such as an adaptive optics system, a method of manufacturing the variable shape mirror, and a method of manufacturing the actuator.

2. Description of the Related Art

A movable mirror and a variable shape mirror of a type to be displaced by an electrostatic attractive force are expected to be applied to various fields utilizing light. For example, the movable mirror and the variable shape mirror each can be utilized as an adaptive optics wavefront correction device to be installed in a fundus inspection apparatus, an astronomical telescope, or the like. As a representative example of such a movable mirror whose reflective surface is displaced by an electrostatic attractive force, there is known a measure of enabling movement by using two parallel plate electrodes, but this parallel plate type has a disadvantage in that the moving amount is small.

In contrast, in recent years, a variable shape mirror that uses a comb electrode structure and can achieve a larger moving amount has been proposed. An example thereof is disclosed in U.S. Pat. No. 6,384,952. In this variable shape mirror, a support portion that supports a comb electrode on a movable side and a support portion that supports a comb electrode on a fixed side are respectively located on upper and lower sides in a direction perpendicular to the support portions. An elastic member for movably supporting the movable portion is manufactured using Cu plating. The movable comb electrode and the fixed comb electrode are opposed to each other, and are arranged so as to be alternately arrayed with a distance. With this, an electrode overlapping area larger than that in the parallel plate type can be achieved. Therefore, a larger electrostatic attractive force can be generated between the movable comb electrode and the fixed comb electrode, and thus, a moving amount of a connecting portion connected to a reflective portion can be increased.

Further, in Japanese Patent Application Laid-Open No. 2010-008613, there is disclosed an SOI substrate formed by coupling an active layer and a substrate layer via a BOX layer. Further, in Japanese Patent Application Laid-Open No. 2010-008613, there is proposed an electrostatic comb actuator structure in which a movable comb electrode, a fixed comb electrode, and an elastic body are formed on an active layer of the SOI substrate. In this structure, the movable portion is driven in a substrate plane direction.

In the related-art electrostatic vertical comb electrode type variable shape mirror having the structure disclosed in U.S. Pat. No. 6,384,952 described above, the elastic member of an electrostatic comb actuator is formed of a Cu plated film, and an elastic member having more excellent spring characteristics is demanded.

In Japanese Patent Application Laid-Open No. 2010-008613, as an elastic member of an electrostatic comb actuator formed of the SOI substrate, an active layer having a thickness of about 30 μm is disclosed. The active layer is formed of monocrystalline silicon, and thus, when used as the elastic member, has more excellent material characteristics than the Cu plated film disclosed in U.S. Pat. No. 6,384,952. When the active layer of the SOI substrate is used as the elastic member, patterning of the active layer of the SOI substrate into a shape of the elastic member, forming an opening that passes through a substrate layer in an area including the elastic member on the substrate layer side to expose the BOX layer, and removing the BOX layer to release the elastic member formed of the active layer are necessary.

Further, when a case is considered where an elastic member formed of an active layer of an SOI substrate is applied to a structure in which a movable portion of an electrostatic comb actuator is displaced in a direction perpendicular to the SOI substrate as in the mirror disclosed in U.S. Pat. No. 6,384,952, the following is assumed: in order to increase an amount of displacement of the movable portion, it is necessary to reduce a thickness of the elastic member formed of the active layer as much as possible; when the opening that passes through the substrate layer of the electrostatic comb actuator is formed by etching, the BOX layer is used as an etching stopper layer, and thus, it is better for the BOX layer to be thick; and, when the active layer and the substrate layer of the electrostatic comb actuator are electrically isolated from each other by the BOX layer, it is better for the BOX layer to be thick. From the reasons described above, in a structure in which the movable portion of the electrostatic comb actuator is displaced in the direction perpendicular to the SOI substrate, a structure is desired in which the elastic member of the electrostatic comb actuator is thin and the BOX layer is thick.

However, in this case, after a step of forming the opening that passes through the substrate layer adjacent to the BOX layer in contact with the active layer of the elastic member until a process of removing the BOX layer for the purpose of releasing the elastic member, there is a concern that deflection due to film stress of the BOX layer in contact with the active layer to be the elastic member may cause stress concentration on a root of the elastic member to increase the possibility of breakage of the elastic member. Therefore, in a variable shape mirror using an electrostatic comb actuator in which a movable portion is displaced in the direction perpendicular to an SOI substrate, when an active layer of the SOI substrate is used as an elastic member, a structure in which breakage of the elastic member is inhibited is demanded.

### SUMMARY OF THE INVENTION

In view of the above-mentioned problem, according to one embodiment of the present invention, there is provided an actuator, including: a supporting member; a plurality of fixed comb electrodes formed on the supporting member and extending from the supporting member; a movable member; an elastic member configured to connect the movable member and the supporting member to each other; and a plurality of movable comb electrodes formed on the movable member, extending from the movable member substantially in parallel with the plurality of fixed comb electrodes, and engaged with the plurality of fixed comb electrodes with gaps therebetween, respectively, a surface of the movable member having the plurality of movable comb electrodes formed thereon and a surface of the supporting member having the plurality of fixed comb electrodes formed thereon being arranged substantially in parallel with movable directions of the movable member, in which the plurality of movable comb electrodes are substantially in parallel with one another, and in which a portion of the elastic member, which is located at a beginning of the extension from the movable member, is substantially in parallel with the plurality of movable comb electrodes.

Further features of the present invention will become apparent from the following description of exemplary embodiments with reference to the attached drawings.

### BRIEF DESCRIPTION OF THE DRAWINGS

FIG. 1A and FIG. 1B are plan views for illustrating an electrostatic comb actuator according to an embodiment of the present invention.

FIG. 2A, FIG. 2B, FIG. 2C, FIG. 2D, FIG. 2E, FIG. 2F, and FIG. 2G are sectional views for illustrating a method of manufacturing the electrostatic comb actuator according to the embodiment.

FIG. 3A, FIG. 3B, FIG. 3C, and FIG. 3D are illustrations of a result of a simulation of stress concentration on elastic members in accordance with a finite element method.

FIG. 4A, FIG. 4B, FIG. 40, FIG. 4D, and FIG. 4E are a plan view of a variable shape mirror and sectional views for illustrating a method of manufacturing the same according to the embodiment.

FIG. 5A, FIG. 5B, FIG. 5C, and FIG. 5D are sectional views for illustrating a method of driving the electrostatic comb actuator according to the present invention.

FIG. 6 is a schematic view of an adaptive optics system and an ophthalmological apparatus using the same according to the present invention.

### DESCRIPTION OF THE EMBODIMENTS

Preferred embodiments of the present invention will now be described in detail in accordance with the accompanying drawings.

In the present invention, in an electrostatic comb actuator used in a variable shape mirror or the like, a plurality of movable comb electrodes extending from a movable member are substantially in parallel with one another, and a portion of an elastic member connecting the movable member and a supporting member to each other, which is located at the beginning of the extension from the movable member, is substantially in parallel with the movable comb electrodes. This can reduce the following concern. In a manufacturing process of the electrostatic comb actuator, for example, a direction of deflection of a BOX layer in contact with a movable comb electrode formed in an SOI substrate in an extending direction and a direction of deflection of the BOX layer in contact with the elastic member in an extending direction intersect each other, which may result in stress concentration on a root of the elastic member to break the elastic member. However, according to the present invention, such a concern can be reduced.

Exemplary structures of the present invention and actions and effects of the present invention are described based on embodiments of the present invention.

(Variable Shape Mirror)

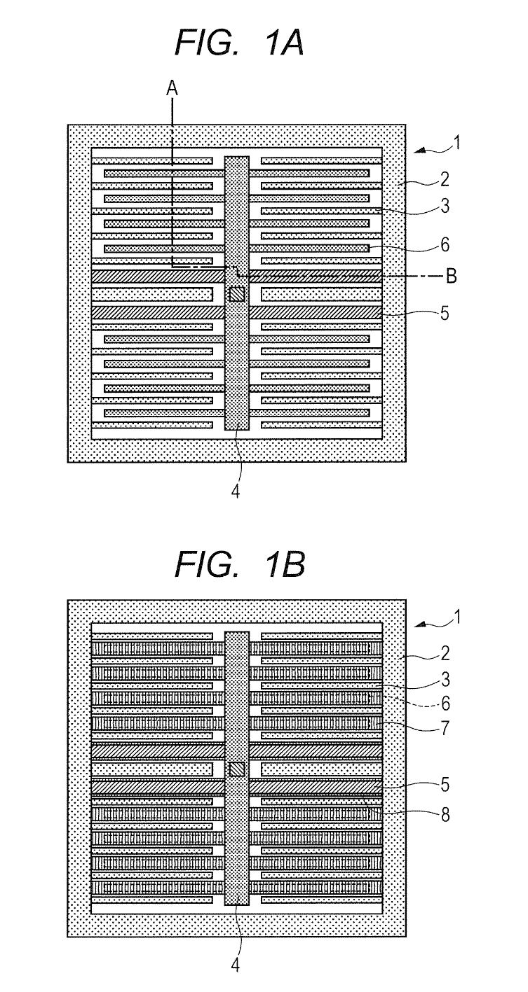

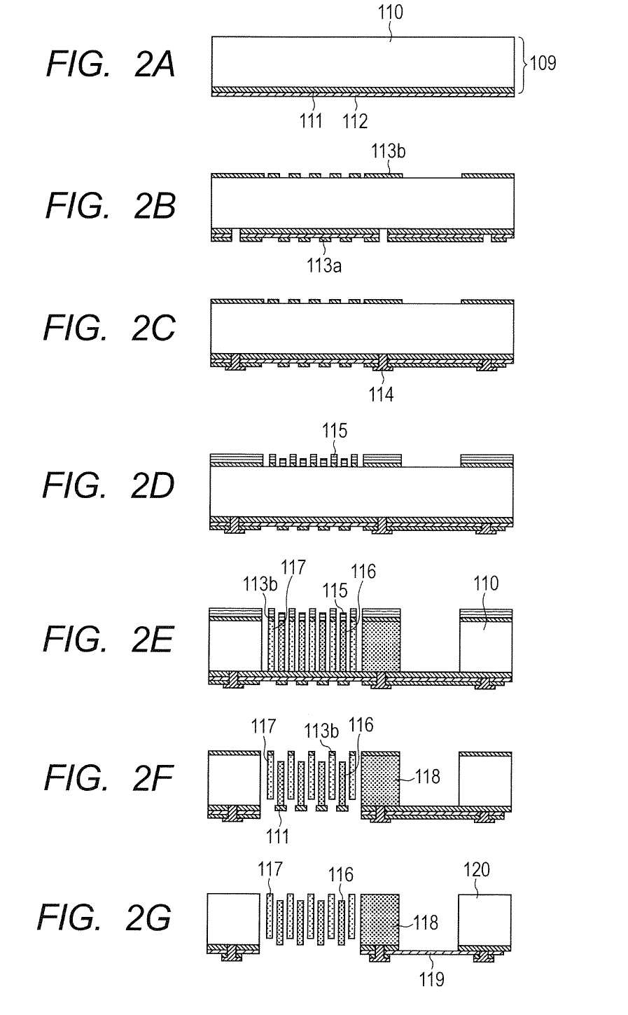

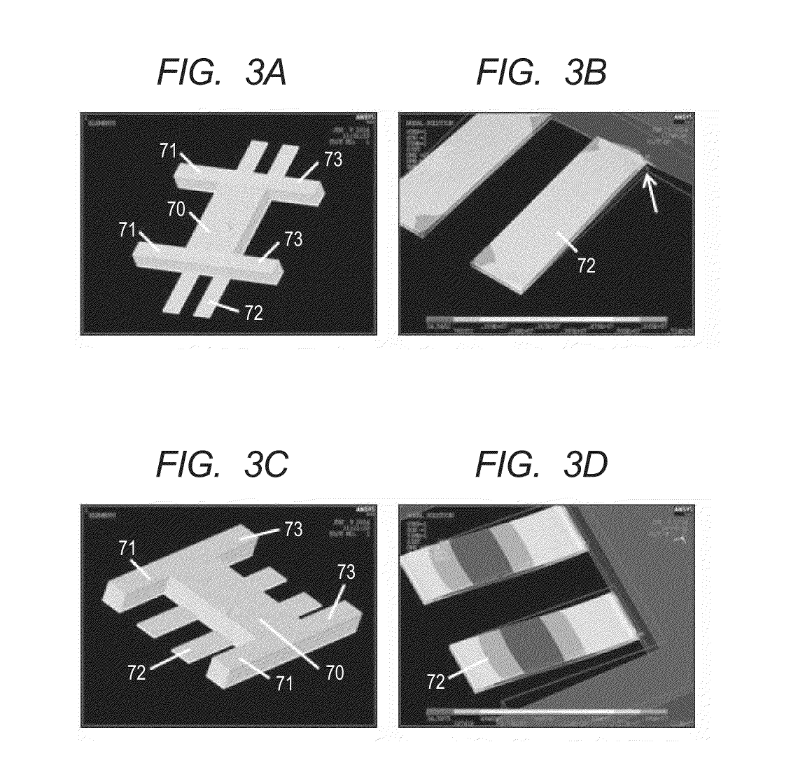

First, an actuator of a variable shape mirror according to an embodiment of the present invention is described with reference to FIG. 1A and FIG. 1B, FIG. 2A to FIG. 2G, and FIG. 3A to FIG. 3D. An actuator array of the variable shape mirror according to this embodiment includes a plurality of electrostatic comb actuators which are displaced to an upper side in a direction vertical (perpendicular) to a mirror surface. A stroke of the displacement (maximum amount of displacement) is relatively small and is, for example, 20 μm, but there is an advantage that the amount of displacement can be finely controlled. FIG. 1A is a plan view seen from a rear surface side of one electrostatic comb actuator of an electrostatic comb actuator array. FIG. 1B is a plan view seen from the rear surface side of the electrostatic comb actuator 1 in the middle of manufacture thereof. As illustrated in FIG. 1A, the variable shape mirror according to this embodiment includes a supporting member 2, and includes the following structural elements: fixed comb electrodes 3 formed on the supporting member 2 and extending in directions in parallel with an upper surface of the supporting member 2 (surface in parallel with the sheet of FIG. 1A and FIG. 1B); a mirror member, one surface thereof being a reflective surface; and a movable member 4 connected to a surface of the mirror member on a side opposite to the reflective surface. The variable shape mirror further includes a plurality of elastic members 5 configured to connect the movable member 4 and the supporting member 2 to each other, and movable comb electrodes 6 formed on the movable member 4, extending in directions in parallel with the fixed comb electrodes 3, and engaged with the fixed comb electrodes 3 with gaps therebetween, respectively.

In the structure described above, surfaces of the movable member 4 having the movable comb electrodes 6 formed thereon and surfaces of the supporting member 2 having the fixed comb electrodes 3 formed thereon are arranged along movable directions of the movable member 4. Further, all the movable comb electrodes 6 extending from the movable member 4 are substantially in parallel with one another, and the movable comb electrodes 6 and portions of all the elastic members 5 connecting the movable member 4 and the supporting member 2 to each other, which are located at the beginnings of the extensions from the movable member 4, are substantially in parallel with one another. In this case, “substantially in parallel” means that an angle formed therebetween falls, for example, within the range of from 0° or more to 5° or less, preferably 1° or less, and optimally 0°.

As can be seen from FIG. 1B for illustrating the electrostatic comb actuator 1 in the middle of manufacture thereof, a pattern 7 formed of the BOX layer in contact with the movable comb electrodes 6 and a pattern 8 formed of the BOX layer in contact with rear sides of the elastic members 5 are substantially in parallel with each other. Therefore, the electrostatic comb actuator 1 in the variable shape mirror according to this embodiment can obtain the following advantage. A direction of deflection of the pattern 7 of the BOX layer in contact with the movable comb electrodes 6 and a direction of deflection of the pattern 8 of the BOX layer in contact with the rear sides of the elastic members 5 are substantially in parallel with each other and do not intersect each other, and thus, in the elastic members 5, stress concentration in a twisting direction with respect to the extending directions is less liable to occur. This enables inhibition of breakage of the elastic members 5 even if the elastic members 5 are relatively thin. In this case, the pattern 7 formed of the BOX layer in contact with the movable comb electrodes 6 and the pattern 8 formed of the BOX layer in contact with the rear sides of the elastic members 5 are substantially in parallel with each other throughout lengths thereof. However, the elastic members 5 may have the shape of cranks in which the portions located at the beginnings of the extensions from the movable member 4 are substantially in parallel with one another and the remaining portions include portions that are not in parallel with one another. However, as can be seen from FIG. 3B and FIG. 3D to be described below, it can be said to be preferred that the portions of the elastic members 5 located at the beginnings of the extensions from the supporting member 2 be also substantially in parallel with the movable comb electrodes.

FIG. 3A to FIG. 3D are illustrations of a result of a simulation using Sample 1 and Sample 2 in which extending directions of movable comb electrodes 71 and elastic members 72 are different from each other in an actuator of a variable shape mirror. This is a result of a simulation of stress concentration on the elastic members of the actuator in accordance with a finite element method. As illustrated in FIG. 3A, Sample 1 had a simple structure including a movable portion 70, movable comb electrodes 71 and 73, and the elastic members 72, and an arrangement was made so that extending directions of the movable comb electrodes 71 and 73 and of the elastic members 72 were perpendicular to each other. As illustrated in FIG. 3C, Sample 2 had a simple structure including the movable portion 70, the movable comb electrodes 71 and 73, and the elastic members 72, and an arrangement was made so that the extending directions of the movable comb electrodes 71 and 73 and of the elastic members 72 were in parallel with each other.

In performing the simulation, a commercial available software (manufactured by ANSYS, Inc.) capable of conducting analysis in accordance with the finite element method was used. Conditions of the simulation were as follows. Note that, for the purpose of clarifying influence of the arrangement of the movable comb electrodes and 73 and the elastic members 72 on the stress concentration on the elastic members 72, the same amount of surface load was applied to upper surfaces of movable comb electrodes 71 on one side and lower surfaces of movable comb electrodes 73 on another side with respect to the movable portion 70.
Dimensions of movable portion: L (length) 600 μm, (width) 300 μm, T (thickness) 100 μm
Dimensions of movable comb electrode: L 300 μm, W 100 μm, T 100 μm
Elastic member: 300 μm, W 100 μm, T 10 μm
Material: monocrystalline silicon (Young's modulus 130 GPa, Poisson's ratio 0.28)
Restraint surface: section of spring (elastic member) on a side opposite to surface connected to movable portion

FIG. 3B is an illustration of the result of the simulation obtained for Sample 1. In FIG. 3B, the magnitude of the concentrated stress is shown with contour lines. From the result of the simulation illustrated in FIG. 3B, it can be seen that stress of the maximum strength is concentrated on an outside end portion of a root of the elastic member on the movable portion side (portion shown by the arrow).

Next, FIG. 3D is an illustration of the result of the simulation obtained for Sample 2. Also in FIG. 3D, similarly to the case of FIG. 3B, the magnitude of the concentrated stress is shown with contour lines. From the result of the simulation illustrated in FIG. 3D, for Sample 2 corresponding to this embodiment, stress of the maximum strength was dispersed on sides of the elastic members 72 in contact with the movable portion 70. Compared with the case of FIG. 3B as a comparative example, the stress of the maximum strength on the elastic members 72 was ⅓ or less. Therefore, it was confirmed that, from the result of the simulation illustrated in FIG. 3A to FIG. 3D, in Sample 2 corresponding to this embodiment, the stress of the maximum strength leading to breakage of the elastic members 72 can be reduced compared with the case of Sample 1 as a comparative example.

Next, a method of manufacturing an electrostatic comb actuator 120 of a variable shape mirror according to this embodiment is described with reference to FIG. 2A to FIG. 2G. FIG. 2A to FIG. 2G are sectional views corresponding to a sectional view taken along the line A-B of FIG. 1A, and are illustrations of a method of manufacturing the structure illustrated in FIG. 1A. In the following, a case in which a plurality of electrostatic comb actuators are formed at the same time on an SOI substrate is described taking only one electrostatic comb actuator 120 as an example.

First, as illustrated in FIG. 2A, an SOI substrate 109 including a substrate layer 110, a BOX layer 111, and an SOI layer (silicon layer) 112 is prepared. The substrate layer 110, the BOX layer 111, and the SOI layer 112 have thicknesses of, for example, 200 μm, 2 μm, and 2 μm, respectively. Next, as illustrated in FIG. 2B, patterns of insulating layers 113 (113a and 113b) are formed on both surfaces of the SOI substrate 109, respectively. Specifically, after silicon oxide (SiO₂) formed by thermal oxidation is used to form the insulating layers 113, resist patterns (not shown) are formed, and the insulating layers 113 are etched with the resist patterns being used as masks, resulting in the patterns (113a and 113b). In etching the insulating layers 113, for example, plasma etching using tetrafluoromethane (CF₄), difluoromethane (CH₂F₂), or trifluoromethane (CHF₃), all of which are chlorofluorocarbon-based gases, is used. Those chlorofluorocarbon-based gases may be used alone or under a state of being mixed with another chlorofluorocarbon-based gas, or being mixed with an inert gas such as argon (Ar) or helium (He).

Then, as illustrated in FIG. 2C, through electrodes 114 each having a contact hole pattern are formed. Specifically, first, a resist pattern (not shown) is formed on a rear surface of the SOI substrate 109. The SOI layer 112 and the BOX layer 111 are etched to form through holes with the resist pattern being used as a mask. Further, after a chromium (Cr) film and a gold (Au) film serving as materials of the electrodes are stacked, a resist pattern (not shown) is formed. The gold (Au) film and the chromium (Cr) film are etched with the resist pattern being used as a mask.

Then, as illustrated in FIG. 2D, a mask for forming the shape of the comb electrode is formed. A resist pattern 115 is formed on a surface of the SOI substrate 109 on the substrate layer 110 side, and the insulating layer 113b on the surface of the substrate layer 110 is etched and patterned. In etching the insulating layer 113b, plasma etching using a chlorofluorocarbon-based gas exemplified in the step illustrated in FIG. 2B used.

Then, as illustrated in FIG. 2E, the movable comb electrodes 116 and the fixed comb electrodes 117 are formed from the substrate layer 110. In this step, the substrate layer 110 is etched with the resist pattern 115 formed as illustrated in FIG. 2D and the insulating layer 113b being used as masks. In this step, in order to form the desired shape of the comb electrode, inductively-coupled-plasma-reactive-ion-etching (ICP-RIE) that enables deep etching in a direction perpendicular to the surface of the substrate layer or the like is used. Through use of ICP-RIE, a fine comb electrode structure having a high aspect ratio can be formed.

Then, as illustrated in FIG. 2F, a level difference is formed in each comb electrode by etching the movable comb electrodes 116 and the fixed comb electrodes 117 for each surface thereof. In order to form a level difference of the fixed comb electrodes 117, the active layer 112 is etched with the insulating layer (SiO₂) 113a on the rear surface being used as a mask. Then, the BOX layer 111 is etched with the etched and patterned active layer 112 being used as a mask. Further, silicon (Si) of the fixed comb electrodes 117 is etched from the rear surface side to a depth of, for example, 20 μm with the etched and patterned BOX layer 111 being used as a mask.

Further, in order to form a level difference of the movable comb electrodes 116, after the resist pattern 115 on the front surface is separated, silicon (Si) of the movable comb electrodes 116 is etched from the front surface side to a depth of, for example, 20 μm with the insulating layer (SiO₂) 113b on the front surface being used as a mask. In etching the silicon (Si) layer and the insulating layer, plasma etching using a chlorofluorocarbon-based gas exemplified with reference to FIG. 2B, ICP-RIE exemplified with reference to FIG. 2D, or the like is used. In the step of forming the level difference of the comb teeth, by forming a level difference between lower surfaces of the fixed comb electrodes 117 and upper surfaces of the movable comb electrodes 116, a comb electrode structure for the electrostatic comb actuator to be displaced by applying a voltage thereto is formed. A drive principle by the comb electrode structure is described later.

FIG. 1B corresponds to a plan view of a rear surface of the electrostatic comb actuator 120 in FIG. 2F. With reference to FIG. 1B, the movable comb electrodes 6 and the elastic members 5 are substantially in parallel with each other. Therefore, the patterns 7 and 8 of the BOX layer corresponding to the BOX layer 111 on lower surfaces of the movable comb electrodes 116 and the BOX layer 111 on upper surfaces of elastic members 119 in FIG. 2F are substantially in parallel with each other. Therefore, deflection of the pattern 7 of the BOX layer and deflection of the pattern 8 of the BOX layer that are caused due to film stress of the BOX layer are in directions substantially in parallel with each other and the directions do not intersect each other, and thus, as in the result of the simulation illustrated in FIG. 3A to FIG. 3D described above, breakage of the elastic members 119 can be inhibited.

Then, as illustrated in FIG. 2G, by etching exposed portions of the BOX layer 111 and of the insulating layer 113 using, for example, buffered hydrofluoric acid (BHF) to release the movable comb electrodes 116 and the elastic members 119, the electrostatic comb actuator 120 is manufactured. Note that, the actuator array and the manufacturing method thereof described above are only exemplary, and the present invention is not limited thereto. With regard to the electrostatic comb actuator 1, the array is, for example, a triangular lattice, and array pitches are, for example, 1,000 μm.

The electrostatic comb actuator of the variable shape mirror described above can inhibit breakage of the elastic member for the reason described above. Further, when the electrostatic comb actuator is manufactured, yield can be improved and manufacturing costs can be reduced. Further, in the electrostatic comb actuator 1 according to this embodiment, all the movable comb electrodes 6 and the elastic members 5 are substantially in parallel with one another. Therefore, compared with, for example, a layout in which the extending direction of the movable comb electrodes and the extending direction of the elastic member are perpendicular to each other, the electrostatic comb actuator can be more compactly laid out with a larger comb electrode region.

(Method of Manufacturing Variable Shape Mirror)

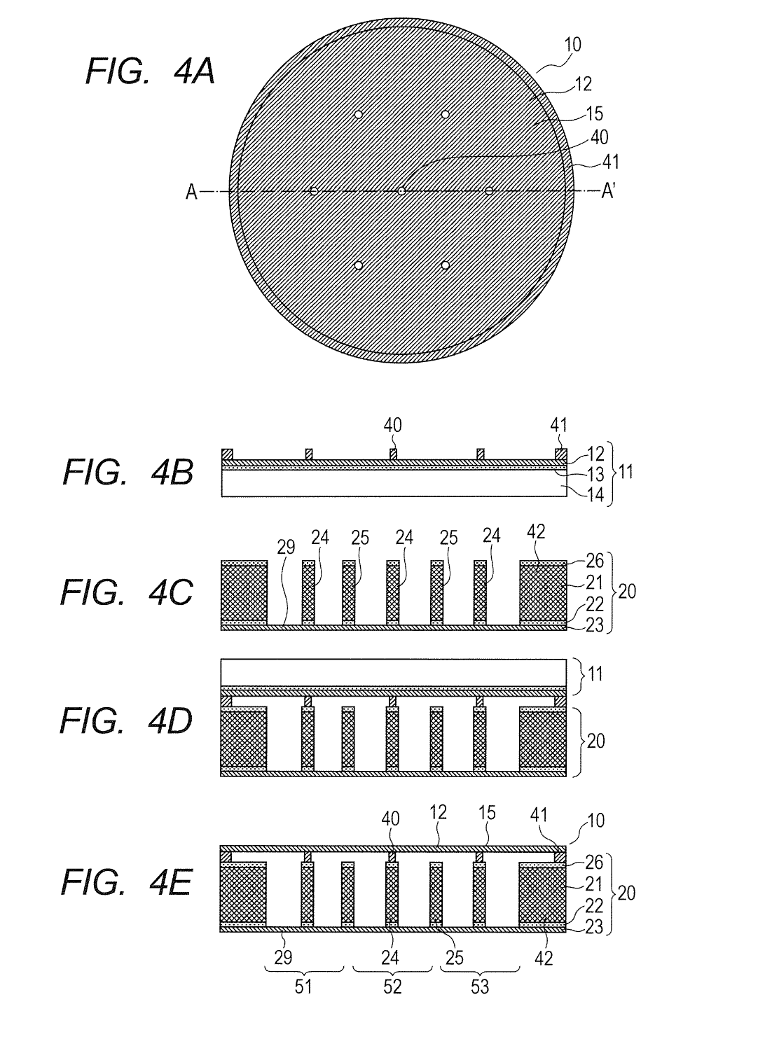

Next, a method of manufacturing a variable shape mirror according to an embodiment of the present invention is described with reference to FIG. 4A to FIG. 4E. FIG. 4A is a plan view of a variable shape mirror 10 according to this embodiment, and FIG. 4B to FIG. 4E are process sectional views taken along the line A-A′ of FIG. 4A. In the method of manufacturing the variable shape mirror 10 according to this embodiment, an active layer 12 of an SOI substrate 11 is a mirror base to be transferred onto an electrostatic comb actuator array 20.

First, as illustrated in FIG. 4B, as a first substrate including three layers of an active layer, an insulator layer (BOX layer), and a substrate layer, for example, the SOI substrate 11 is prepared. The SOI substrate 11 includes, for example, the active layer 12 formed of silicon, a substrate layer 14, and a BOX layer 13 of silicon oxide formed therebetween. Further, posts 40 serving as portions connecting to movable members of the electrostatic comb actuator and a circumferential connecting portion 41 serving as a portion connecting to a fixed member on a periphery are formed on the active layer 12.

Then, as illustrated in FIG. 4C, the electrostatic comb actuator array 20 for displacing the active layer 12 as the mirror base is prepared. As described above, the electrostatic comb actuator array 20 includes supporting members 25, a circumferential supporting member 42, and fixed comb electrodes (not shown) formed on the supporting members and extending in directions in parallel with upper surfaces of the supporting members. The electrostatic comb actuator array 20 further includes movable members 24, elastic members 29 configured to connect the movable members 24 and the supporting members 25 and 42 to each other, and movable comb electrodes (not shown) formed on the movable members 24, extending in parallel with the fixed comb electrodes, and engaged with the fixed comb electrodes with gaps therebetween, respectively. Surfaces of the movable members 24 having the movable comb electrodes formed thereon and surfaces of the supporting members 25 and 42 having the fixed comb electrodes formed thereon are arranged along movable directions of the movable members 24.

Further, all the movable comb electrodes extending from the movable members 24 are substantially in parallel with one another. Further, the movable comb electrodes and portions of all the elastic members 29 connecting the movable members 24 and the supporting members 25 and 42 to each other, which are located at the beginnings of extensions from the movable members 24, are substantially in parallel with each other. Connecting portions (not shown) for connection to the posts 40 and 41 formed on the mirror base 12 are formed on the movable members 24 and the circumferential supporting member 42.

Then, as illustrated in FIG. 4D, the first substrate 11 from which the mirror base 12 is formed and the first actuator array 20 are bonded together via the posts 40 and the circumferential connecting portion 41. As the posts 40 and the circumferential connecting portion 41, for example, Au bumps are used. Further, as the connecting portions formed the movable members 24 and the circumferential supporting member 42, for example, Au pads are used. In this bonding, for example, Au—Au surface activated bonding is used. In this method, the bonding is performed after the surfaces of the Au bumps and the Au pads are activated by removing organic matters thereof by Ar plasma.

Note that, room temperature surface activated bonding is used as the bonding method according to this embodiment, but the present invention is not limited thereto. In this case, the first substrate 11 from which the mirror base 12 is formed and the first actuator substrate 20 are, when bonded together, aligned with each other by aligning alignment marks (not shown) formed on the first actuator substrate with alignment marks (not shown) formed on the first substrate. The alignment in the bonding can be performed with an accuracy of ±0.5 μm or less, and thus, the plurality of movable portions 24 can be arranged in a state of being aligned with the mirror base 12 with high accuracy.

Then, as illustrated in FIG. 4E, the substrate layer 14 and the BOX layer 13 of the SOI substrate 11 serving as the first substrate are removed. This can form a structure in which the mirror base formed of the active layer 12 is connected to the electrostatic comb actuator array 20. The substrate layer 14 is removed by, for example, silicon dry etching. An end of the etching is controlled by plasma emission spectroscopy, and the BOX layer 13 of the SOI substrate 11 is used as an etching stopper layer. In this silicon dry etching, by adopting conditions with which an etching selectivity ratio between the BOX layer 13 serving as the etching stopper layer and the substrate layer is high, the BOX layer 13 protects the active layer 12, and thus, the active layer 12 is not etched. In this case, the substrate layer 14 may be removed by wet etching using an aqueous solution of tetramethylammonium hydroxide (TMAH) or the like.

Then, the BOX layer 13 is removed by, for example, wet etching using buffered hydrofluoric acid (BHF). In this case, the active layer 12 under the BOX layer 13 has a high etching selectivity ratio with respect to the BOX layer 13, and thus, is hardly etched. Therefore, the BOX layer 13 can be removed without damaging the mirror base 12 (active layer 12). Note that, the BOX layer 13 may be removed by, other than this, dry etching using vapor hydrofluoric acid.

Then, a reflectivity of the variable shape mirror may be improved by forming a reflective film 15 on the mirror base. The reflective film is made of, for example, Au, and, as an adhesive layer, for example, Ti may be used.

As described above, in the variable shape mirror according to this embodiment, breakage of an elastic member of an electrostatic comb actuator is inhibited, and thus, yield when the variable shape mirror is manufactured can be improved to reduce manufacturing costs.

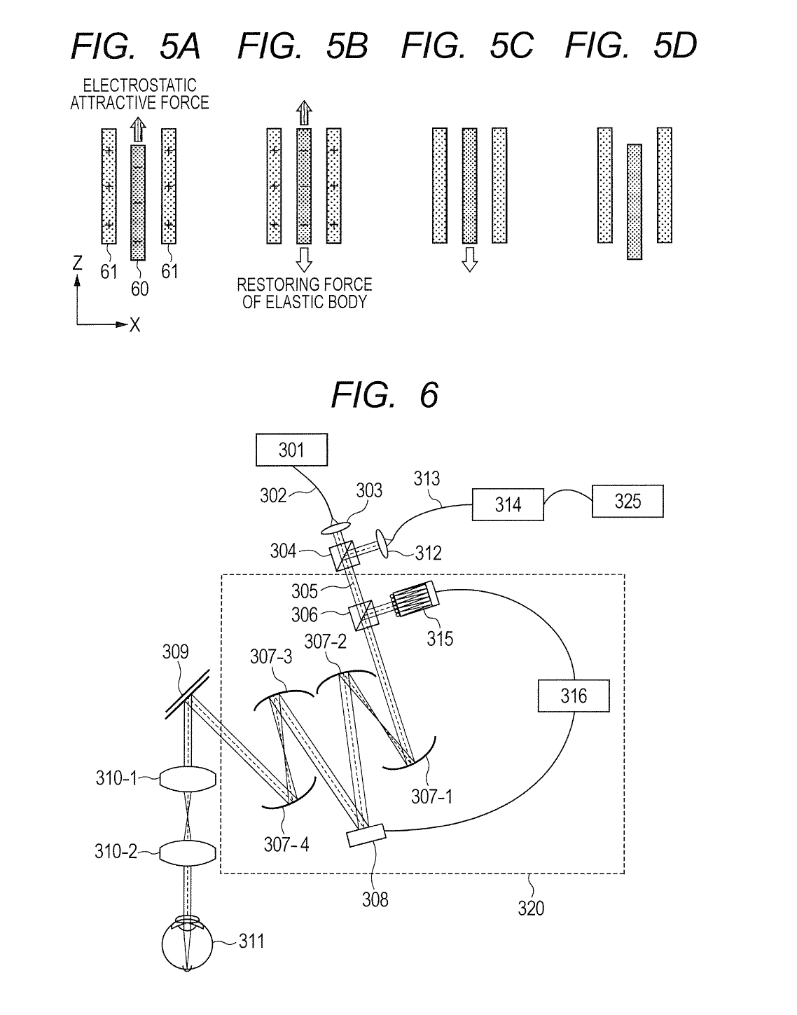

FIG. 5A to FIG. 5D are sectional views for illustrating a drive principle of the electrostatic comb actuator according to the present invention, and, as a structure of an electrostatic comb actuator having electrode pairs, only a movable comb electrode 60 and fixed comb electrodes 61 are illustrated for the sake of simplicity. As illustrated in FIG. 5A, immediately after a voltage is applied, by applying charges of opposite polarities to the movable comb electrode 60 and the fixed comb electrodes 61, respectively, an electrostatic attractive force is generated between the comb electrodes, which displaces the movable comb electrode 60 to a plus side in directions perpendicular to the substrate (Z directions). In other words, by the electrostatic attractive force generated between the comb electrodes, the movable portion (not shown in FIG. 5A to FIG. 5D) that supports the movable comb electrode 60 can be driven. Note that, due to the electrostatic attractive force, the movable comb electrode 60 attempts to come closer to the fixed comb electrodes 61, but, with regard to the horizontal direction (X directions), a substantially uniform electrostatic attractive force is applied on right and left sides of the electrodes, and thus, the movable comb electrode 60 is displaced to an upper side in the perpendicular direction.

In a balanced state illustrated in FIG. 5B, the elastic body (not shown in FIG. 5A to FIG. 5D) has the function of, when the movable comb electrode 60 is displaced by the electrostatic attractive force, stopping the movable comb electrode 60 at a position at which the electrostatic attractive force and a restoring force of the elastic body are balanced. As illustrated in FIG. 5C, after the voltage is released, the electrostatic attractive force between the comb electrodes is released, and thus, the balance between the electrostatic attractive force and the restoring force of the elastic body is lost, and the restoring force of the elastic body acts on the movable comb electrode 60. As illustrated in FIG. 5D, after the displacement, the restoring force of the elastic body returns the movable comb electrode 60 to its initial position. The polarities of voltages applied to the movable comb electrode 60 and the fixed comb electrodes 61 (polarities of applied charges) may be opposite to those illustrated in FIG. 5A to FIG. 5D. The displacement of the movable portion is controlled in accordance with the voltages applied to the movable comb electrode and the fixed comb electrodes.

In this case, an electrostatic attractive force Fz in the perpendicular direction that acts when a potential difference is applied between the movable comb electrode 60 and the fixed comb electrodes 61 is represented by the following formula 1:

Fz=[(ε₀·N·h)/(2g)]·(Vm−Vf)² (Formula 1),

where ε₀ represents a permittivity of vacuum, N represents the number of gaps between the comb electrodes, h represents an overlapping length between the movable comb electrode and the fixed comb electrodes, Vm represents a potential of the movable comb electrode, Vf represents a potential of the fixed comb electrodes, and g represents a width of the gap between the comb electrodes.

Therefore, in the actuator array 20 illustrated in FIG. 4A to FIG. 4E, the fixed comb electrodes (not shown) are grounded, and a voltage is applied to the movable comb electrodes (not shown) connected to the plurality of movable members 24 via wiring (not shown) individually connected to the movable members 24. In this way, the plurality of movable members 24 can be individually displaced to the mirror base side. Note that, the actuator has seven posts 40 in FIG. 4A. That is, in FIG. 4A, a structure is illustrated in which seven electrostatic comb actuators are connected to the variable shape mirror 10 having a continuous reflective surface. However, this structure is only exemplary, and, by increasing the number of the actuators, a more complicated mirror surface shape can be realized with higher accuracy.

(Ophthalmological Apparatus)

An adaptive optics system that uses the variable shape mirror described above as a wavefront correction device that compensates for an optical aberration is described with a scanning laser ophthalmoscope (hereinafter described as “SLO apparatus”) as an example. The SLO apparatus is an ophthalmological apparatus configured to irradiate a fundus with light so as to enable observation of a photoreceptor, a retinal nerve fiber layer, hemodynamics, or the like.

FIG. 6 is an illustration of a schematic configuration of the SLO apparatus of this embodiment. Light emitted from a light source 301 travels through a single-mode optical fiber 302 and passes through a collimator 303 to become a collimated light beam. The collimated light beam is transmitted through a beam splitter 304, which serves as a light splitting unit, as measurement light 305 to be guided to an adaptive optics system 320. The wavelength of the light source 301 for emitting, for example, laser light is not particularly limited, but particularly for fundus imaging, the wavelength of about 800 nm to about 1,500 nm (for example, wavelength of 850 nm or less) is suitably used for preventing dazzling of a subject and for maintaining the resolution. The adaptive optics system 320 includes a beam splitter 306 serving as a light splitting unit, a wavefront sensor (aberration measuring unit) 315, a variable shape mirror that forms a reflective optical modulation element (wavefront correction device) 308, and reflective mirrors 307-1 to 307-4 for guiding the light to those members. The respective reflective mirrors 307 are placed so that at least the pupil of the eye to be inspected, the wavefront sensor 315, and the variable shape mirror 308 have an optically conjugate relationship.

The light that has passed through the adaptive optics system 320 scanned by a light scanning portion 309 one-dimensionally or two-dimensionally. The measurement light scanned by the light scanning portion 309 is radiated to an eye 311 to be inspected through eyepiece lenses 310-1 and 310-2. By adjusting the positions of the eyepiece lenses 310-1 and 310-2, optimum irradiation can be performed in accordance with the visibility of the eye 311 to be inspected. In this case, a lens is used in the eyepiece part, but a spherical mirror or the like may be used instead.

The measurement light radiated to the eye 311 to be inspected is reflected or scattered by a fundus (retina). The light reflected or scattered at the fundus of the eye 311 to be inspected travels, in an opposite direction, a passage similar to that during entrance, and is partially reflected by the beam splitter 306 to enter the wavefront sensor 315. Thus, this partially reflected light is used to measure the wavefront of the light beam. As the wavefront sensor 315, a known Shack-Hartmann sensor can be used. The reflected or scattered light that has transmitted through the beam splitter 306 is partially reflected by the beam splitter 304 to be guided to a light intensity sensor 314 through a collimator 312 and an optical fiber 313. Light that has entered the light intensity sensor 314 is converted into an electrical signal to be processed into a fundus image by an image processing unit 325.

The wavefront sensor 315 is connected to an adaptive optics controller 316 serving as a control unit to transmit the measurement result of the wavefront of the received light beam to the adaptive optics controller 316. The adaptive optics controller 316 is connected to the variable shape mirror 308 including the electrostatic comb actuator according to the present invention, and the variable shape mirror 308 is deformed into a shape instructed by the adaptive optics controller 316.

The adaptive optics controller 316 calculates, based on the wavefront obtained from the wavefront sensor 315, a mirror shape that enables correction into a wavefront with no aberration. Then, in order to reproduce the shape in the variable shape mirror 308, a necessary application voltage difference for each of the comb electrodes is calculated and sent to the variable shape mirror 308. In the variable shape mirror 308, a potential difference sent from the adaptive optics controller 316 is applied between the movable comb electrode and the fixed comb electrode, to thereby deform the mirror surface into a predetermined shape.

The measurement of the wavefront by the wavefront sensor 315, transmission of the wavefront to the adaptive optics controller 316, and instruction by the adaptive optics controller 316 to the variable shape mirror for correction of the aberration as described above are repeatedly processed to be feed-back controlled to constantly obtain an optimum wavefront. Note that, it is only necessary that the variable shape mirror that forms the reflective optical modulation element is arranged so as to correct a wavefront aberration of at least one of measurement light or return light.

The adaptive optics system according to this embodiment described above inhibits breakage of the spring of the electrostatic comb actuator, and thus, manufacturing costs when the adaptive optics system is manufactured can be reduced.

According to the one embodiment of the present invention, in the actuator having the structure described above, stress concentration on the elastic members of the actuator may be reduced in the manufacturing process thereof, and thus, breakage of the elastic members may be inhibited

While the present invention has been described with reference to exemplary embodiments, it is to be understood that the invention not limited to the disclosed exemplary embodiments. The scope of the following claims is to be accorded the broadest interpretation so as to encompass all such modifications and equivalent structures and functions.

This application claims the benefit of Japanese Patent Application No. 2015-013791, filed Jan. 27, 2015, which is hereby incorporated by reference herein in its entirety.

What is claimed is:

1. An actuator comprising:
a supporting member;
a plurality of fixed comb electrodes formed on the supporting member and extending from the supporting member;
a movable member;
an elastic member configured to connect the movable member and the supporting member to each other; and
a plurality of movable comb electrodes formed on the movable member, extending from the movable member substantially in parallel with the plurality of fixed comb electrodes, and engaged with the plurality of fixed comb electrodes with gaps therebetween, respectively,
a surface of the movable member having the plurality of movable comb electrodes formed thereon and a surface of the supporting member having the plurality of fixed comb electrodes formed thereon being arranged substantially in parallel with movable directions of the movable member,
wherein the plurality of movable comb electrodes are substantially in parallel with one another, and
wherein a portion of the elastic member, which is located at a beginning of extension from the movable member, is substantially in parallel with the plurality of movable comb electrodes.

2. The actuator according to claim 1, wherein a plurality of the elastic members are provided, and portions of the plurality of the elastic members, which are located at beginnings of extensions from the movable member, are substantially in parallel with the plurality of movable comb electrodes.

3. The actuator according to claim 1, wherein the elastic member is substantially in parallel with the plurality of movable comb electrodes throughout a length of the elastic member.

4. The actuator according to claim 1, wherein a plurality of the elastic members are provided, and portions of all of the plurality of the elastic members, which are located at beginnings of extensions from the movable member, are substantially in parallel with the plurality of movable comb electrodes throughout lengths of the plurality of the elastic members.

5. A variable shape mirror comprising:
the actuator according to claim 1; and
a mirror member, one surface of the mirror member being a reflective surface,
wherein the movable member of the actuator is connected to a surface of the mirror member on a side opposite to the reflective surface.

6. An ophthalmological apparatus configured to obtain an image of an eye to be inspected, comprising:
a reflective optical modulation element configured to correct wavefront aberration of at least one of measurement light and return light;
an aberration measurement unit configured to measure an aberration caused at the eye to be inspected; and
a control unit configured to control the reflective optical modulation element based on a result of the measurement by the aberration measurement unit,
the reflective optical modulation element comprising the variable shape mirror according to claim 5.

7. An adaptive optics system configured to correct a wavefront aberration, comprising:
a reflective optical modulation element configured to correct a wavefront aberration of incident light;
an aberration measurement unit configured to measure the wavefront aberration of the incident light; and
a control unit configured to control the reflective optical modulation element based on a result of the measurement by the aberration measurement unit,
the reflective optical modulation element comprising the variable shape mirror according to claim 5.

8. A method of manufacturing an actuator, comprising:
preparing a first substrate comprising three layers of a silicon layer, an insulator layer, and a substrate layer; and
forming, on a second substrate comprising three layers of a silicon layer, an insulator layer, and a substrate layer, a connecting portion and an actuator, the actuator comprising an electrode pair having a comb electrode structure for displacing the connecting portion in a direction perpendicular to a reflective surface of a mirror member,
the actuator comprising:
a supporting member;
a plurality of fixed comb electrodes formed on the supporting member and extending from the supporting member;
a movable member movable in the perpendicular direction;
an elastic member configured to connect the movable member and the supporting member to each other; and
a plurality of movable comb electrodes formed on the movable member, extending from the movable member substantially in parallel with the plurality of fixed comb electrodes, and engaged with the plurality of fixed comb electrodes with gaps therebetween, respectively,
a surface of the movable member having the plurality of movable comb electrodes formed thereon and a surface of the supporting member having the plurality of fixed comb electrodes formed thereon being arranged substantially in parallel with the perpendicular direction,
wherein, in the forming a connecting portion and an actuator:
all patterns comprising the insulator layer of the second substrate in contact with the plurality of movable comb electrodes extending from the movable member are substantially in parallel with one another; and
a pattern in contact with the plurality of movable comb electrodes and a portion of a pattern comprising the insulator layer of the second substrate in contact with the elastic member configured to connect the movable member and the supporting member to each other, the portion being located at a beginning of extension from the movable member, are substantially in parallel with each other.

9. A method of manufacturing a variable shape mirror, comprising:
the steps of the method of manufacturing an actuator of claim 8;
bonding together the first substrate and the second substrate via the connecting portion; and
removing the substrate layer and the insulator layer of the first substrate.
